(19) United States
(12) Patent Application Publication
(10) Pub. No.: US 2021/0379016 A1
(43) Pub. Date: Dec. 9, 2021

(54) THERAPEUTICS AND METHODS OF TREATMENT OF ANGIOTENSIN-CONVERTING ENZYME 2 ASSOCIATED CONDITIONS

(71) Applicant: Board of Supervisors of Louisiana State University and Agricultural and Mechanical College, Baton Rouge, LA (US)

(72) Inventors: Shenuarin BHUIYAN, Shreveport, LA (US); Christopher KEVIL, Shreveport, LA (US); Anthony Wayne ORR, Benton, LA (US); Chowdhury S ABDULLAH, Shreveport, LA (US); Richa Aishwarya, Shreveport, LA (US)

(73) Assignee: Board of Supervisors of Louisiana State University and Agricultural and Mechanical College, Baton Rouge, LA (US)

(21) Appl. No.: 17/341,249

(22) Filed: Jun. 7, 2021

Related U.S. Application Data
(60) Provisional application No. 63/035,300, filed on Jun. 5, 2020.

(51) Int. Cl. A61K 31/4015; A61K 45/06; A61K 31/55; A61K 31/5375; A61K 31/341; A61K 31/445; A61K 31/57

(52) U.S. Cl. CPC A61K 31/4015; A61K 45/06; A61K 31/55; A61K 31/57; A61K 31/341; A61K 31/445; A61K 31/5375

(57) ABSTRACT

Therapeutics and methods of treating one of an angiotensin-converting enzyme 2 (ACE2) associated condition and an ACE2 associated pre-condition patient comprising administering to the patient a pharmaceutically therapeutic dose of a therapeutic, wherein the therapeutic includes either a Sigmar1 antagonist or any pharmaceutically acceptable salt, solvate, or prodrug thereof, or a Sigmar1 enhancer, or any pharmaceutically acceptable salt, solvate, or prodrug thereof. A method of treating coronavirus disease 2019 (COVID-19) patient comprising administering to the patient a pharmaceutically therapeutic dose of a therapeutic, wherein the therapeutic includes a Sigmar1 enhancer, or any pharmaceutically acceptable salt, solvate, or prodrug thereof.

### CROSS REFERENCE TO RELATED APPLICATIONS/PRIORITY

The present invention claims priority to United States Provisional Patent Application No. 63/035,300 filed Jun. 5, 2020, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under grant HL145753 awarded by the National Institutes of Health. The government has certain rights in the invention.

### BACKGROUND

The severe acute respiratory syndrome coronavirus 2 (SARS-CoV-2), which causes COVID-19 (coronavirus disease 2019), has infected over 157,000,000 people and killed over 3,000,000 people worldwide in a little over one year, with the number continuing to rise. Vaccines have been developed for some strains of the virus, but newly mutated strains continue to appear. For these reasons there is a pressing need to discover successful treatments for COVID-19.

### SUMMARY

Wherefore, it is an object of the present invention to overcome the above-mentioned shortcomings and drawbacks associated with the current technology.

The presently disclosed invention is related to therapeutics and methods of treating one of an angiotensin-converting enzyme 2 (ACE2) associated condition and an ACE2 associated pre-condition patient comprising administering to the patient a pharmaceutically therapeutic dose of a therapeutic, wherein the therapeutic includes either a Sigmar1 antagonist or any pharmaceutically acceptable salt, solvate, or prodrug thereof, or a Sigmar1 enhancer, or any pharmaceutically acceptable salt, solvate, or prodrug thereof. According to a further embodiment, the one of the ACE2 associated condition and the ACE2 associated pre-condition is associated with an elevated ACE2 level and the therapeutic includes a Sigmar1 antagonist. According to a further embodiment, the ACE2 associated condition is one of mice ventricular tachycardia and fibrillation, cardiac arrhythmia, colonic levels in ulcerative colitis. According to a further embodiment, the one of the ACE2 associated condition and the ACE2 associated pre-condition is associated with a reduced ACE2 level and the therapeutic includes a Sigmar1 enhancer. According to a further embodiment, the one of the ACE2 associated condition and the ACE2 associated pre-condition is one of coronavirus disease 2019 (COVID-19), myocardial infarction damage, myocardial fibrosis, perivascular fibrosis, glomerulosclerosis, renal deposition of type I and III collagen and fibronectin, increased albuminuria; hypertension; heart failure, coronary artery disease, peripheral vascular disease, diabetic kidney; renal damage induced by diabetes/hypertension, Alzheimer's disease (AD), Parkinson's disease (PD), Huntington's disease, multiple sclerosis (MS), and, with reduced small bowel ACE2 level, Crohn's disease. According to a further embodiment, the therapeutic includes the Sigmar1 antagonist, or any pharmaceutically acceptable salt, solvate, or prodrug thereof. According to a further embodiment, the Sigmar1 antagonist includes one of AC927, AHD1, AZ66, BD1008, BD-1047, BD1060, BD1063, BD1067, BMY-14802, CM156, E-5842, Haloperidol, LR132, LR172, MS-377, NE-100, Panamesine, Phenothiazines, Progesterone, Rimcazole, E-52862, Sertraline, UMB100, UMB101, UMB103, UMB116, YZ-011, YZ-069, and YZ-185. According to a further embodiment, the therapeutic includes the Sigmar1 enhancer, or any pharmaceutically acceptable salt, solvate, or prodrug thereof. According to a further embodiment, the Sigmar1 enhancer one of increases Sigmar1 function, increases Sigmar1 expression, and increases both increases Sigmar1 function and Sigmar1 expression. According to a further embodiment, the Sigmar1 enhancer is one of a Sigmar1 Agonists and a Sigmar1 positive allosteric modulator (PAM). According to a further embodiment, the Sigmar1 enhancer is a Sigmar1 Agonist. According to a further embodiment, the Sigmar1 Agonist is one of PRE-084, ANAVEX2-73, donepezil, fluvoxamine, citalopram, amitriptyline, L-687,384, SA-4503, dextromethorphan, dimethyltryptamine, (+)-pentazocine, and opipramol, 3-MeO-PCP, afobazole, BD1031, BD1052, memantine, and pentoxyverine. According to a further embodiment, the Sigmar1 enhancer is a Sigmar1 PAM. According to a further embodiment, the Sigmar1 PAM is one of Methylphenylpiracetam and SOMCL-668. According to a further embodiment, the Sigmar1 enhancer increases Sigmar1 expression. According to a further embodiment, the Sigmar1 enhancer is a gene therapeutic. According to a further embodiment, the gene therapeutic is a recombinant Sigmar1 adenovirus infection in a tissue with a lowered ACE2 level. According to a further embodiment, the gene therapeutic is a SIGMAR1 gene promotor. According to a further embodiment, the gene therapeutic is one of an oligonucleotide therapy, a CAR-T therapy, a AAV transgene delivery, a gene editor, CRISPR-Cas9, and a universal donor cell therapy.

The presently disclosed invention is further related to therapeutics and methods of treating coronavirus disease 2019 (COVID-19) patient comprising administering to the patient a pharmaceutically therapeutic dose of a therapeutic, wherein the therapeutic includes a Sigmar1 enhancer, or any pharmaceutically acceptable salt, solvate, or prodrug thereof.

A method of decreasing ACE2 activity comprising administering a Sigmar1 antagonist.

A method of increasing ACE2 activity comprising administering a Sigmar1 enhancer.

The present invention relates to pharmaceutical compositions of a therapeutic (e.g., a Sigmar1 antagonist or a Sigmar1 enhancer), or a pharmaceutically acceptable salt, solvate, ester, amide, clathrate, stereoisomer, enantiomer, prodrug or analogs thereof, and use of these compositions for the treatment of a ACE2 associated condition, including COVID-19.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is a ACE2 associated condition.

In certain embodiments, the ACE2 associated condition is mild to moderate ACE2 associated condition.

In further embodiments, the ACE2 associated condition is moderate to severe ACE2 associated condition.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the ACE2 associated condition.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., Sigmar1 antagonist or a Sigmar1 enhancer, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a ACE2 associated condition). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a micrograph of immunostaining showing the expression of Sigmar1 (green) and ACE2 (red) in mouse lung. Sigmar1 co-localized with ACE2 (yellow) in mouse lung, evidencing Sigmar1 interaction with ACE2.

FIG. 2 shows Sigmar1 controlling ACE2 expression. Western blot analysis showing decreased ACE2 and TMPRSS2 expression in lung and kidney cells lysates from Sigmar1 global knockout mouse (Sigmar1^{-/-}).

FIG. 3 shows Sigmar1 dependent ACE2 expression in human aortic endothelial cells. Western blot analysis showing decreased ACE2 expression in Sigmar1-siRNA knockdown human aortic endothelial cells.

FIG. 4 shows Sigmar1 ligands controlling ACE2 expression. HEK-293 cells, which abundantly express the ACE2, were treated with Sigmar1 ligands Sertraline and NE-100 for 24 hours. Treatment with Sertraline and NE-100 significantly decreased the ACE2 expression in HEK-293 cells.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In addition, the invention does not require that all the advantageous features and all the advantages of any of the embodiments need to be incorporated into every embodiment of the invention.

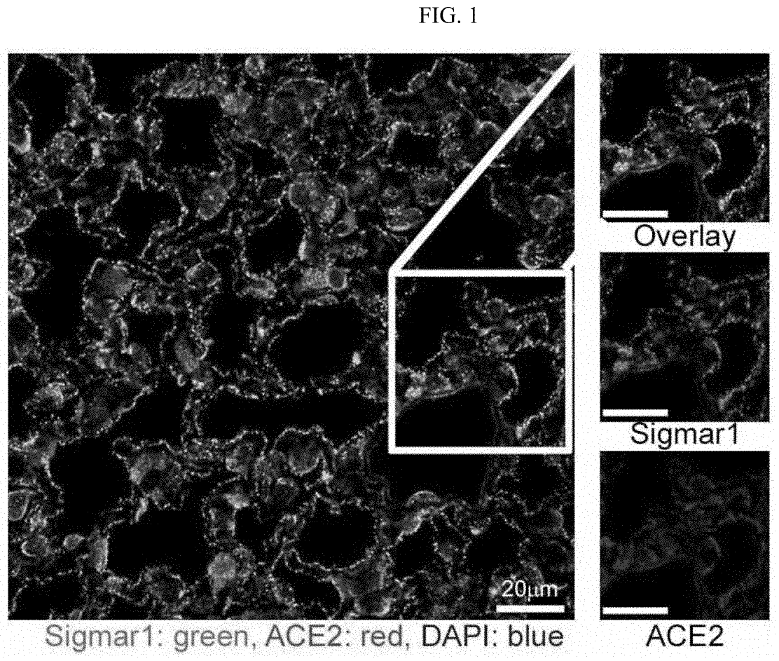

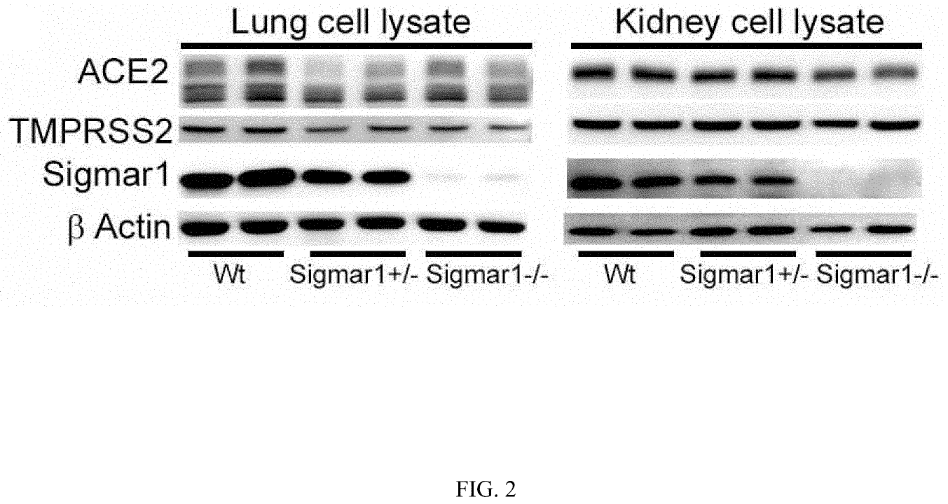

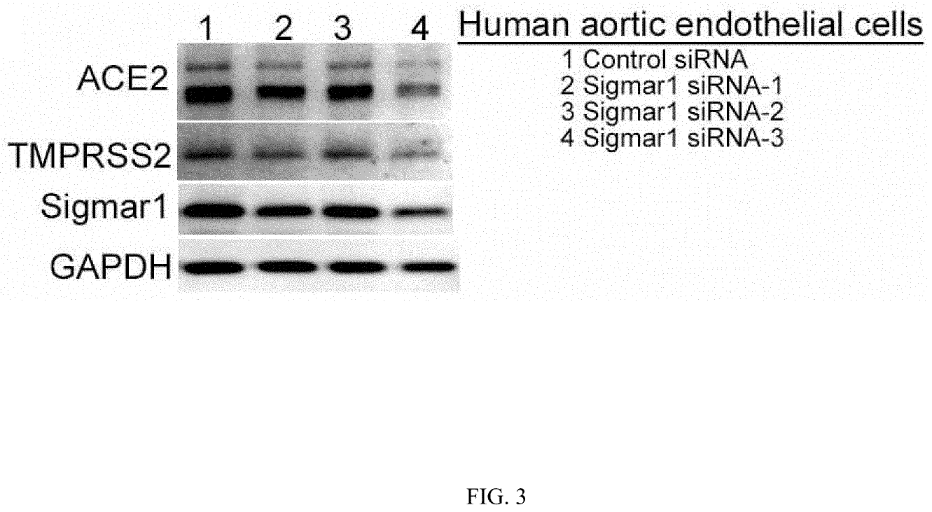

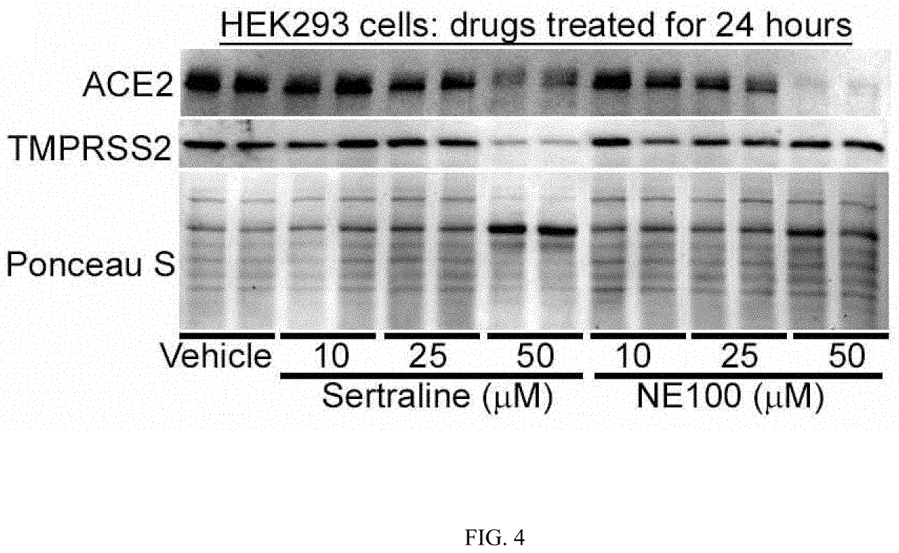

Turning now to FIGS. 1-4, a brief description concerning the various components of the present invention will now be briefly discussed. The experiments shown evidence that Sigmar1 ligands can be used to control cellular ACE2 expression and activity.

This disclosure is believed to be the first experiment to demonstrate that Sigma 1 receptor (Sigmar1) expression in the lung, kidney, and aortic cells control angiotensin-converting enzyme 2 (ACE2) expression. The inventors observed the presence of Sigmar1 and ACE2 in the lung using immunostaining. The inventors found Sigmar1 co-localization with ACE2 in the lung cells, evidencing Sigmar1 interaction with ACE2. To further explore whether Sigmar1 expression alters the ACE2 expression, the inventors performed Western blot analysis for ACE2 and transmembrane protease serine 2 (TMPRSS2) in lung and kidney cells lysates from Sigmar1 global knockout mouse (Sigmar1^{-/-}). The inventors found a significantly decreased expression of ACE2 and TMPRSS2 in the cell lysates from Sigmar1^{-/-}. Further to confirm these data, the inventors knocked down Sigmar1 in human aortic endothelial cells by siRNA transfection and also observed decreased expression of ACE2. Finally, the inventors treated the HEK-293 cells, which abundantly express the ACE2 with Sigmar1 ligands Sertraline and NE-100. Treatment with Sertraline and NE-100 significantly decreased the ACE2 expression in HEK-293 cells. All these experiments evidence a molecular function of Sigmar1 in regulating ACE2 expression and, therefore, identifies a new tool in developing treatment regimens for diseases associated with altered ACE2 expression and activity.

The coronavirus disease 2019 (COVID-19), caused by severe acute respiratory syndrome coronavirus 2 (SARS-CoV-2), binds to ACE2 for cell entry, mediated via the viral surface spike glycoprotein (S protein). Subsequently, the S protein is cleaved by the transmembrane protease serine 2 TMPRSS2. Therefore, the Sigmar1 ligands can be therapeutically used to treat the COVID-19 by alleviating the cytokine storm. Moreover, ligands targeting Sigmar1 can also be used for the treatment of cardiovascular diseases associated with ACE2 altered activity such as heart failure, coronary artery disease, and peripheral vascular disease. Moreover, Sigmar1 ligand-dependent ACE2 regulation can be therapeutically used in other conditions associated with ACE2 involvement.

Sigmar1 targeted with selective drugs can be therapeutically used for any diseases associated with ACE2 altered activity and expression.

The coronavirus disease 2019 (COVID-19) is caused by a novel severe acute respiratory syndrome coronavirus 2 (SARS-CoV-2) that shows an unusually high transmission rate and unique clinical characteristics. The clinical manifestation includes acute respiratory distress syndrome, metabolic acidosis, septic shock, coagulation dysfunction, and organ failures such as liver, kidney, and heart failure. Despite extensive ongoing studies, the pathological mechanism remaining unclear, and there is no effective medication for the treatment of COVID-19. SARS-CoV-2 has been suggested to bind to angiotensin-converting enzyme 2 (ACE2) for cell entry, mediated via the viral surface spike glycoprotein (S protein). Subsequently, the S protein is cleaved by the transmembrane protease serine 2 (TMPRSS2). In our current study, we found that Sigma 1 receptor (Sigmar1) regulates the expression of ACE2 and TMPRSS2 in the lung, kidney, and aortic cells. We found that the treatment of cells with the Sigmar1 ligands (e.g., Sertraline and NE-100) decreases the ACE2 expression. Hence, Sigmar1 can be targeted with selective ligands to modulate ACE2 expression and activity to prevent diseases involving ACE2, including COVID-19, hypertension, Crohn's diseases and neurovascular diseases.

Sigmar1 ligands may be used to regulate the expression of ACE2 in COVID-19. Additionally, the Sigmar1 dependent regulation of ACE2 expression can be used to treat any diseases having altered ACE2 activity such as Crohn's disease, hypertension, heart failure and neurodegenerative diseases, just for example. ACE2 level adjustment has been shown to improve many ACE related conditions, and, even if not cure, can prime the patient for greater efficacy of secondary therapeutics.

Conditions that have been shown to exacerbated by elevated ACE2 levels and treated, at least in part, by reducing ACE2 levels include ventricular tachycardia and fibrillation, cardiac arrhythmia, and, with elevated ACE2 colonic level, ulcerative colitis.

Conditions that have been shown to exacerbated by reduced ACE2 levels and treated, at least in part, by elevating ACE2 levels include coronavirus disease 2019 (COVID-19), myocardial infarction damage, myocardial fibrosis, perivascular fibrosis, glomerulosclerosis, renal deposition of type I and III collagen and fibronectin, increased albuminuria; hypertension; heart failure, coronary artery disease, peripheral vascular disease, diabetic kidney; renal damage induced by diabetes/hypertension, Alzheimer's disease (AD), Parkinson's disease (PD), Huntington's disease, multiple sclerosis (MS), and, with reduced small bowel ACE2 level, Crohn's disease.

Based on the inventors' experimental results, the level of ACE2 may be moderated in a patient with an ACE2 associated condition by raising ACE2 levels through administration of a Sigmar1 enhancer for conditions of reduced ACE2 levels or by lowering ACE2 levels through the administration of a Sigmar1 antagonist for conditions of elevated ACE2 levels, giving medical science new tools to treat deadly diseases.

Exemplary Sigmar1 enhancers and methods of increasing Sigmar1 function include administration of Sigmar1 agonists, such as, PRE-084, ANAVEX2-73, donepezil, fluvoxamine, citalopram, amitriptyline, L-687,384, SA-4503, dextromethorphan, dimethyltryptamine, (+)-pentazocine, and opipramol, 3-MeO-PCP, afobazole, BD1031, BD1052, memantine, pentoxyverine, for example, and/or Sigmar1 positive allosteric modulators (PAMs), such as methylphenylpiracetam, and SOMCL-668, for example. Additionally, exemplary Sigmar1 enhancers and methods for increasing Sigmar1 expression include administration of gene therapeutics such as recombinant Sigmar1 adenovirus infection in the cardiac tissue, administration of SIGMAR1 gene promotor. Additional SIGMAR1 gene therapeutics and/or gene promotors to increase the expression of SIGMAR1 include administration of include oligonucleotide therapies, CAR-T therapies, AAV transgene delivery, gene editing (CRISPR-Cas9, for example), and universal donor cell therapies. Unless otherwise specified, SIGMR1 refers to the human SIGMAR1 gene, hSIGMAR1, with an approximate location on the human genome of located approximately chr9:34,634,722-34,637,844. The disclosed Sigmar1 enhancers include their respective pharmaceutically acceptable salts, solvates, esters, amides, clathrates, stereoisomers, enantiomers, prodrugs and analogs thereof in such disclosure as if fully recited herein.

Exemplary Sigmar1 antagonists include AC927, AHD1, AZ66, BD1008, BD-1047, BD1060, BD1063, BD1067, BMY-14802, CM156, E-5842, Haloperidol, LR132, LR172, MS-377, NE-100, Panamesine, Phenothiazines, Progesterone, Rimcazole, E-52862, Sertraline, UMB100, UMB101, UMB103, UMB116, YZ-011, YZ-069, YZ-185. The disclosed Sigmar1 antagonists include their respective pharmaceutically acceptable salts, solvates, esters, amides, clathrates, stereoisomers, enantiomers, prodrugs and analogs thereof in such disclosure as if fully recited herein. More detailed information regarding a subset of that list follows below.

AC927 or 1-(2-phenylethyl) piperidine is a selective sigma receptor antagonist, with binding affinity of Kᵢ=30±2 nM for Sigmar1 and a chemical structure of:

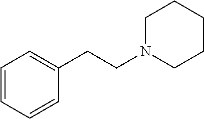 [SMILES: c1ccc(CCN2CCCCC2)cc1]

BD1008 or N-[2-(3,4-dichlorophenyl)ethyl]-N-methyl-1-pyrrolidineethanamine is a selective sigma receptor antagonist, with a binding affinity of Kᵢ=2±1 nM for Sigmar1 and a chemical structure of:

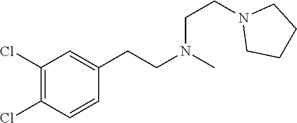 [SMILES: CN(CCc1ccc(Cl)c(Cl)c1)CCN1CCCC1]

BD-1047 is a sigma receptor antagonist, selective for the σ1 subtype and a has chemical structure of

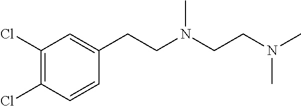 [SMILES: CN(C)CCN(C)CCc1ccc(Cl)c(Cl)c1]

BD106 or N-[2-(3,4-dichlorophenyl)ethyl]-1-pyrrolidineethanamine is a selective sigma receptor antagonist, with a binding affinity of Ki=3±0.1 nM for Sigmar1 and a chemical structure of:

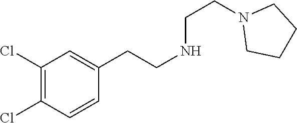 [SMILES: Clc1ccc(CCNCCN2CCCC2)cc1Cl]

BD1063 or 1-[2-(3,4-dichlorophenyl)ethyl]-4-methylpiperazine is a selective sigma receptor antagonist, with a binding affinity of Ki=9±1 nM for Sigmar1 and a chemical structure of:

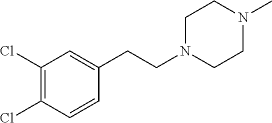 [SMILES: CN1CCN(CCc2ccc(Cl)c(Cl)c2)CC1]

BD1067 or N-[2-(3,4-dichlorophenyl)ethyl]-N-ethyl-1-pyrrolidineethanamine is a selective sigma receptor antagonist, with a binding affinity of Ki=2±0.5 nM for Sigmar1 and a chemical structure of:

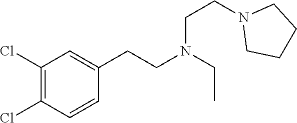 [SMILES: CCN(CCc1ccc(Cl)c(Cl)c1)CCN1CCCC1]

BMY-14802, also known as BMS-181100, is a sigma receptor antagonist and has a chemical structure of:

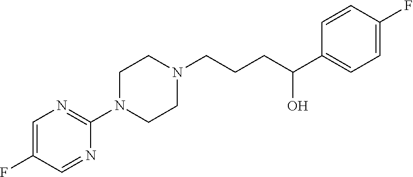 [SMILES: OC(CCCN1CCN(c2ncc(F)cn2)CC1)c1ccc(F)cc1]

CM156 or 3-(4-(4-cyclohexylpiperazin-1-yl) butyl) benzo [d]thiazole-2(3H)-thione is a piperazine based chemical compound with nanomolar affinity for Sigmar1 and a chemical structure of:

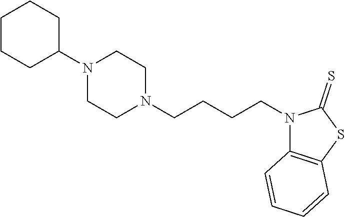 [SMILES: S=c1sc2ccccc2n1CCCCN1CCN(C2CCCCC2)CC1]

Haloperidol acts on Sigmar1 with irreversible inactivation by haloperidol metabolite HPP+ at around 3 nM and a chemical structure of:

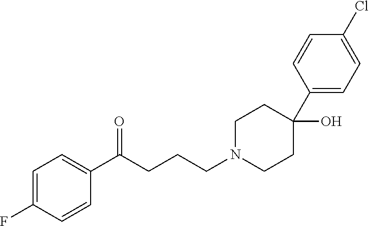 [SMILES: O=C(CCCN1CCC(O)(c2ccc(Cl)cc2)CC1)c1ccc(F)cc1]

LR132 or (+)-3,4-dichloro-N-[(1R,2S)-2-(1-pyrrolidinyl) cyclohexyl] benzeneethanamine is a selective sigma receptor antagonist, with a reported binding affinity of Ki=2±0.1 nM for Sigmar1 and a chemical structure of:

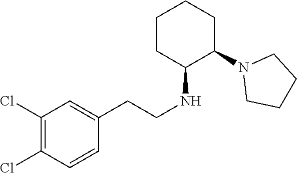 [SMILES: Clc1ccc(CCN[C@H]2CCCC[C@H]2N2CCCC2)cc1Cl]

NE-100 or 4-methoxy-3-(2-phenylethoxy)-N,N-dipropylbenzeneethanamine is a selective Sigmar1 antagonist, with a binding affinity of Ki=1.03±0.01 nM and a chemical structure of:

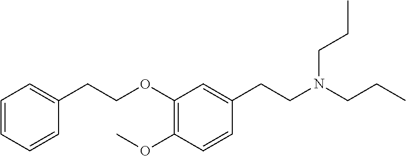 [SMILES: CCCN(CCC)CCc1ccc(OC)c(OCCc2ccccc2)c1]

Panamesine, also known as INN developmental code name EMD-57455, is a sigma receptor antagonist with a sigmar1 IC50=6 nM and a chemical structure of:

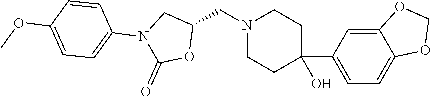 [SMILES: COc1ccc(N2C[C@H](CN3CCC(O)(c4ccc5c(c4)OCO5)CC3)OC2=O)cc1]

Phenothiazines, abbreviated PTZ, is an organic compound that has the formula S(C₆H₄)₂NH, is related to the thiazine-class of heterocyclic compounds, and has a chemical structure of:

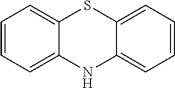 [SMILES: c1ccc2c(c1)Nc1ccccc1S2]

Progesterone (P4) is an endogenous steroid, progestogen sex hormone, and Sigmar1 antagonist, which a chemical structure of:

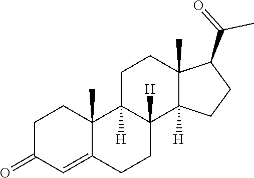

Rimcazole is an antagonist of the sigma receptor and a chemical structure of:

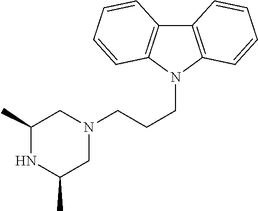 [SMILES: C[C@@H]1CN(CCCn2c3ccccc3c3ccccc32)C[C@H](C)N1]

E-52862, also known as Sigmar1 antagonist (S1A, S1RA), as well as MR-309, is a selective Sigmar1 antagonist, with a binding affinity of Ki=17.0±7.0 nM and a chemical structure of:

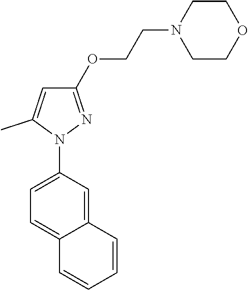

Sertraline is an antagonist of the sigma σ1 receptor, with a Ki=32-57 nM, and a chemical structure of:

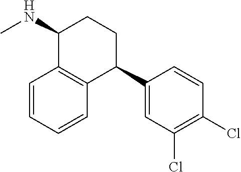 [SMILES: CN[C@H]1CC[C@@H](c2ccc(Cl)c(Cl)c2)c2ccccc21]

Pharmaceutical Compositions: The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that enhance Sigmar1 for Sigmar1 enhancers and those that inhibit Sigmar1 for Sigmar1 antagonists.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration: The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings: The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration: Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery: Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

The pharmaceutical compositions of the invention may be dispensed to the subject under treatment with the help of an applicator. The applicator to be used may depend on the specific medical condition being treated, amount and physical status of the pharmaceutical composition, and choice of those skilled in the art. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the skin surface, liquid in the container is transferred to the skin in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

The pharmaceutical compositions of the invention may be provided to the subject or the medical professional in charge of dispensing the composition to the subject, along with instructional material. The instructional material includes a publication, a recording, a diagram, or any other medium of expression, which may be used to communicate the usefulness of the composition and/or compound used in the practice of the invention in a kit. The instructional material of the kit may, for example, be affixed to a container that contains the compound and/or composition used in the practice of the invention or shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Other routes of administration to the affected area which are contemplated include: transdermal, mucosal, rectal, and vaginal, or topical (for example, in a carrier vehicle, a topical control release patch, in a wound dressing, a hydrocolloid, a foam, or a hydrogel, a cream, a gel, a lotion, an ointment, a liquid crystal emulsion (LCE), and/or a micro-emulsion). An appropriate biological carrier or pharmaceutically acceptable excipient may be used. Compounds administered may, in various embodiments, be racemic, isomerically purified, or isomerically pure.

Transmucosal Administration: Transmucosal administration is carried out using any type of formulation or dosage unit suitable for application to mucosal tissue. For example, the selected active agent may be administered to the buccal mucosa in an adhesive tablet or patch, sublingually administered by placing a solid dosage form under the tongue, lingually administered by placing a solid dosage form on the tongue, administered nasally as droplets or a nasal spray, a non-aerosol liquid formulation, or a dry powder, placed within or near the rectum (“transrectal” formulations), or administered to the urethra as a suppository, ointment, or the like. Application in the oral or nasal cavities are options for high absorption that does not make a first pass in the liver, especially for treatment of ACE2 associated condition locations proximate to the administration, such as the cheek for example.

Transrectal Administration: Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention may be manufactured using conventional processes. The transrectal dosage unit may be fabricated to disintegrate rapidly or over a period of several hours. The time period for complete disintegration may be in the range of from about 10 minutes to about 6 hours, e.g., less than about 3 hours. This can be an option for administration for high absorption that does not make a first pass in the liver, especially for treatment of ACE2 associated condition locations proximate to the administration, such as the gluteal region for example.

Vaginal or Perivaginal Administration. Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention may be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit may be fabricated to disintegrate rapidly or over a period of several hours. The time period for complete disintegration may be in the range of from about 10 minutes to about 6 hours, e.g., less than about 3 hours. This can be an option for administration for high absorption that does not make a first pass in the liver, especially for treatment of ACE2 associated condition locations proximate to the administration, such as the genital region for example.

Topical Formulations: Topical formulations may be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

Transdermal Administration: Transdermal compound administration, which is known to one skilled in the art, involves the delivery of pharmaceutical compounds via percutaneous passage of the compound into the systemic circulation of the patient. Topical administration may also involve the use of transdermal administration such as transdermal patches or iontophoresis devices. Other components may be incorporated into the transdermal patches as well. For example, compositions and/or transdermal patches may be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions may include creams, sprays, lotions, gels, ointments, eye drops, nose drops, ear drops, and the like. In such dosage forms, the compositions of the invention may be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions may contain polyethylene glycol 400. They may be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, may be impregnated with the compositions in solution, lotion, cream, ointment or other such form may also be used for topical application. The compositions may also be applied topically using a transdermal system, such as one of an acrylic-based polymer adhesive with a resinous crosslinking agent impregnated with the composition and laminated to an impermeable backing.

Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir that, in this case, may be either a polymeric matrix as described above, or be a liquid or hydrogel reservoir, or take some other form.

Additional Administration Forms. Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Application Nos. 20030147952, 20030104062, 20030104053, 20030044466, 20030039688, and 20020051820. Additional dosage forms of this invention also include dosage forms as described in PCT Application Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757, such forms incorporated by reference.

Solutions: After a Sigmar1 antagonist or enhancer has been selected, it may be dissolved into a solution. The solution may be an aqueous-based solution, such as water, saline, or the like. In some variations, other fluids and solutions may be appropriate.

Various formulations of saline are known in the art and may be used with the present invention. For example, the saline may be lactated Ringer's solution, acetated Ringer's solution, phosphate buffered saline (PBS), Dulbecco's phosphate buffered saline (D-PBS), Tris-buffered saline (TBS), Hank's balanced salt solution (HBSS), or Standard saline citrate (SSC).

The saline solutions of the present invention are, in certain embodiments, “normal saline” (i.e., a solution of about 0.9% w/v of NaCl). Normal saline has a slightly higher degree of osmolality compared to blood; however, in various embodiments, the saline may be isotonic in the body of a subject such as a human patient. In certain embodiments, “half-normal saline” (i.e., about 0.45% NaCl) or “quarter-normal saline” (i.e., about 0.22% NaCl) may be used with the present invention. Optionally, about 5% dextrose or about 4.5 g/dL of glucose may be included in the saline. In various embodiments, one or more salt, buffer, amino acid and/or antimicrobial agent may be included in the saline.

In various embodiments, a preservative or stabilizer may be included in the composition or solution. For example, the prevention of the action of microorganisms may be brought about by preservatives such as various antibacterial and antifungal agents, including but not limited to parabens (for example, methylparabens, propylparabens), chlorobutanol, phenol, sorbic acid, EDTA, metabisulfite, benzyl alcohol, thimerosal or combinations thereof. Agents that may be included suitable for use include sterile aqueous solutions or dispersions and sterile powders for the extemporaneous preparation of sterile solutions or dispersions (U.S. Pat. No. 5,466,468, specifically incorporated herein by reference in its entirety). In all cases the composition is preferably sterile and must be fluid to facilitate easy injectability. Solutions are preferably stable under the conditions of manufacture and storage and must be preserved against the contaminating action of microorganisms, such as bacteria and fungi. Examples of stabilizers which may be included include buffers, amino acids such as glycine and lysine, carbohydrates such as dextrose, mannose, galactose, fructose, lactose, sucrose, maltose, sorbitol, mannitol, and the like. Appropriate stabilizers or preservatives may be selected according to the route of administration desired. A particle filter or microbe filter may be used, and may be necessary according to the route of administration desired.

The weight ranges of compounds in the solution may vary. For example, in various embodiments, the composition may comprise about 0.1-10 wt %, more preferably 1-5 wt % Sigmar1 antagonist or enhancer, about 1-5 wt % preservative/stabilizer, about 1-5 wt % NaCl, and about 85%-97% water. The ratio of Sigmar1 antagonist or enhancer to water may be varied as needed to achieve the desired treatment of the ACE2 associated condition.

The solution and/or composition may also be sterilized prior to administration. Methods for sterilization are well known in the art and include heating, boiling, pressurizing, filtering, exposure to a sanitizing chemical (for example, chlorination followed by dechlorination or removal of chlorine from solution), aeration, autoclaving, and the like.

The Sigmar1 antagonist or enhancer may be formulated into a solution in any number of ways. For example, it may be solubilized by agitation or by sonication, or other methods known in the art. After the Sigmar1 antagonist or enhancer has been solubilized, it may be administered to a subject in need of treatment of a ACE2 associated condition. In certain embodiments, a Sigmar1 antagonist or enhancer is admixed with a solution in a closed vacuum container, and the combined solutions are then mechanically agitated for 3-5 minutes and held in a thermo-neutral sonicator until use.

In certain embodiments, solutions of the present invention may be a component of an emulsion, such as a water-in-oil or an oil-in-water emulsion, including a lipid emulsion, such as a soybean oil emulsion. Certain emulsions have been described previously for intravenous (da Silva Telles, et al., 2004, Rev. Bras. Anaestesiol Campianas 54(5):2004) or epidural administration (Chai et al. 2008, British J Anesthesia 100:109-115), such described emulsion techniques incorporated by reference herein.

Pharmaceutical compositions of the present invention comprise an effective amount of one or more Sigmar1 antagonist or enhancer dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one Sigmar1 antagonist or enhancer in solution or additional active ingredient will be known to those of skill in the art in light of the present disclosure, as exemplified by “Remington: The Science and Practice of Pharmacy,” 20th Edition (2000), which is incorporated herein by reference in its entirety. Moreover, for animal (for example, human) administration, it will be understood that preparations should meet sterility, pyrogenicity, general safety and purity standards as required by FDA Office of Biological Standards.

In various embodiments, the compositions of the present invention further comprise cyclodextrin. Cyclodextrins are a general class of molecules composed of glucose units connected to form a series of oligosaccharide rings (See Challa et al., 2005, AAPS PharmSciTech 6:E329-E357). In nature, the enzymatic digestion of starch by cyclodextrin glycosyltransferase (CGTase) produces a mixture of cyclodextrins comprised of 6, 7 and 8 anhydroglucose units in the ring structure (α-, β-, and γ-cyclodextrin, respectively). Commercially, cyclodextrins are also produced from starch, but different, more specific enzymes are used. Cyclodextrins have been employed in formulations to facilitate the delivery of cisapride, chloramphenicol, dexamethasone, dextromethoraphan, diphenhydramine, hydrocortisone, itraconazole, and nitroglycerin (Welliver and McDonough, 2007, Sci World J, 7:364-371). In various embodiments, the cyclodextrin of the invention is hydroxypropyl-Beta-cyclodextrin, sulfobutylether-beta-cyclodextrin, alpha-dextrin or combinations thereof. In certain embodiments, cyclodextrin may be used as a solubilizing agent.

In various other embodiments, compositions of the present invention may comprise human serum albumin purified from plasma, or recombinant human serum albumin. In certain embodiments, human serum albumin may be used as a solubilizing agent. In other embodiments, the compositions of the invention may comprise propylene glycol. In other embodiments, the compositions of the invention may comprise perfluorooctyl bromide. In other embodiments, the compositions of the invention may comprise perfluorocarbon. In certain embodiments, perfluorocarbon may be used as a solubilizing agent.

In various embodiments, a preservative or stabilizer may be included in the composition or solution. For example, the prevention of the action of microorganisms may be brought about by preservatives such as various antibacterial and antifungal agents, including but not limited to parabens (for example, methylparabens, propylparabens), chlorobutanol, phenol, sorbic acid, EDTA, metabisulfite, benzyl alcohol, thimerosal or combinations thereof. Agents which may be included suitable for use include sterile aqueous solutions or dispersions and sterile powders for the extemporaneous preparation of sterile solutions or dispersions (U.S. Pat. No. 5,466,468, specifically incorporated herein by reference in its entirety). In all cases the composition is preferably sterile and must be fluid to facilitate easy injectability. Solutions are preferably stable under the conditions of manufacture and storage and must be preserved against the contaminating action of microorganisms, such as bacteria and fungi. Examples of stabilizers which may be included include buffers, amino acids such as glycine and lysine, carbohydrates such as dextrose, mannose, galactose, fructose, lactose, sucrose, maltose, sorbitol, mannitol, etc. Appropriate stabilizers or preservatives may be selected according to the route of administration desired. A particle filter or microbe filter may be used and may be necessary according to the route of administration desired.

Administration of the disclosed compositions in a method of treatment may be achieved in a number of different ways, using methods known in the art. Such methods include, but are not limited to, topically administering solutions, suspensions, creams, pastes, oils, lotions, gels, foam, hydrogel, ointment, liposomes, emulsions, liquid crystal emulsions, and nano-emulsions.

The therapeutic and prophylactic methods of the invention thus encompass the use of pharmaceutical compositions of the invention. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. For example unit dose container may be such that Sigmar1 antagonist or enhancer solution is contained in a crushable sealed ampoule which in turn is enclosed in protective covering on which pressure is applied to crush the ampoule which then releases Sigmar1 antagonist or enhancer solution for percolation through a flint-type tip which capped the ampoule in protective covering. When such packaging configuration is employed, care is taken to leave as little as possible or ideally no headspace in ampoule for any volatile portion of the solution to escape and cause a change in solution composition over a period of shelf life.

Although the description of pharmaceutical compositions provided herein are principally directed to pharmaceutical compositions which are suitable for ethical administration to humans, it will be understood by the skilled artisan that such compositions are generally suitable for administration to animals of all sorts, including mammals. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, vaginal, topical, intranasal, buccal, or another route of administration.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Non-limiting examples of such an additional pharmaceutically active agents are fluorouracil cream, imiquimod cream, ingenol mebutate gel, diclofenac sodium gel, topical retinoids, and tirbanibulin (Klisyri) ointment.

Controlled- or sustained-release formulations of a pharmaceutical composition of the invention may be made using conventional technology.

Formulations of a pharmaceutical composition suitable for topical administration comprise the active ingredient combined with a pharmaceutically acceptable carrier, such as sterile water or sterile isotonic saline. Formulations may be prepared, packaged, or sold in unit dosage form, such as in ampules, crushable or otherwise, or in multi-dose containers containing a preservative. Formulations for topical administration include, but are not limited to, suspensions, solutions, emulsions in oily or aqueous vehicles, solutions, suspensions, creams, pastes, oils, lotions, gels, foam, hydrogel, ointment, liposomes, emulsions, liquid crystal emulsions, nanoemulsions, implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile aqueous or oily suspension or solution. This suspension or solution may be formulated according to the known art, and may comprise, in addition to the active ingredient, additional ingredients such as the dispersing agents, wetting agents, or suspending agents described herein. Such sterile formulations may be prepared using a non-toxic acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other formulations that are useful include those which comprise the active ingredient in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

In some embodiments, the pharmaceutical compositions of the invention may be contained in a crushable ampule irrespective of the route of delivery to the patient.

It is contemplated that any embodiment discussed in this specification may be implemented with respect to any method or composition of the invention, and vice versa. Furthermore, compositions of the invention may be used to achieve methods of the invention.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

Dosing Regimes

The present methods for treating ACE2 associated conditions are carried out by administering a therapeutic for a time and in an amount sufficient to result in decreased or normalized ACE2 level for a condition associated with elevated ACE2 level, or to result in increased or normalized ACE2 level for a condition associated with decreased ACE2 level.

The amount and frequency of administration of the compositions can vary depending on, for example, what is being administered, the state of the patient, and the manner of administration. In therapeutic applications, compositions can be administered to a patient suffering from ACE2 associated condition in an amount sufficient to relieve or least partially relieve the symptoms of the ACE2 associated condition and its complications. The dosage is likely to depend on such variables as the type and extent of progression of the ACE2 associated condition, the severity of the ACE2 associated condition, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the ACE2 associated condition or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits: Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the ACE2 associated condition.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

Wherefore, I/We claim:

1. A method of treating one of an angiotensin-converting enzyme 2 (ACE2) associated condition and an ACE2 associated pre-condition patient comprising:
administering to the patient a pharmaceutically therapeutic dose of a therapeutic,
wherein the therapeutic includes either
a) a Sigmar1 antagonist or any pharmaceutically acceptable salt, solvate, or prodrug thereof, or
b) a Sigmar1 enhancer, or any pharmaceutically acceptable salt, solvate, or prodrug thereof.

2. The method of claim 1 wherein the one of the ACE2 associated condition and the ACE2 associated pre-condition is associated with an elevated ACE2 level and the therapeutic includes a Sigmar1 antagonist.

3. The method of claim 4 wherein the ACE2 associated condition is one of mice ventricular tachycardia and fibrillation, cardiac arrhythmia, colonic levels in ulcerative colitis.

4. The method of claim 1 wherein the one of the ACE2 associated condition and the ACE2 associated pre-condition is associated with a reduced ACE2 level and the therapeutic includes a Sigmar1 enhancer.

5. The method of claim 1 wherein the one of the ACE2 associated condition and the ACE2 associated pre-condition is one of coronavirus disease 2019 (COVID-19), myocardial infarction damage, myocardial fibrosis, perivascular fibrosis, glomerulosclerosis, renal deposition of type I and III collagen and fibronectin, increased albuminuria; hypertension; heart failure, coronary artery disease, peripheral vascular disease, diabetic kidney; renal damage induced by diabetes/hypertension, Alzheimer's disease (AD), Parkinson's disease (PD), Huntington's disease, multiple sclerosis (MS), and, with reduced small bowel ACE2 level, Crohn's disease.

6. The method of claim 1 wherein the therapeutic includes the Sigmar1 antagonist, or any pharmaceutically acceptable salt, solvate, or prodrug thereof.

7. The method of claim 2 wherein the Sigmar1 antagonist includes one of AC927, AHD1, AZ66, BD1008, BD-1047, BD1060, BD1063, BD1067, BMY-14802, CM156, E-5842, Haloperidol, LR132, LR172, MS-377, NE-100, Panamesine, Phenothiazines, Progesterone, Rimcazole, E-52862, Sertraline, UMB100, UMB101, UMB103, UMB116, YZ-011, YZ-069, and YZ-185.

8. The method of claim 1, wherein the therapeutic includes the Sigmar1 enhancer, or any pharmaceutically acceptable salt, solvate, or prodrug thereof.

9. The method of claim 1, wherein the Sigmar1 enhancer one of increases Sigmar1 function, increases Sigmar1 expression, and increases both increases Sigmar1 function and Sigmar1 expression.

10. The method of claim 9, wherein the Sigmar1 enhancer is one of a Sigmar1 Agonists and a Sigmar1 positive allosteric modulator (PAM).

11. The method of claim 10, wherein the Sigmar1 enhancer is a Sigmar1 Agonist.

12. The method of claim 11, wherein the Sigmar1 Agonist is one of PRE-084, ANAVEX2-73, donepezil, fluvoxamine, citalopram, amitriptyline, L-687,384, SA-4503, dextromethorphan, dimethyltryptamine, (+)-pentazocine, and opipramol, 3-MeO-PCP, afobazole, BD1031, BD1052, memantine, and pentoxyverine.

13. The method of claim 10, wherein the Sigmar1 enhancer is a Sigmar1 PAM.

14. The method of claim 13, wherein the Sigmar1 PAM is one of Methylphenylpiracetam and SOMCL-668.

15. The method of claim 9, wherein the Sigmar1 enhancer increases Sigmar1 expression.

16. The method of claim 15, wherein the Sigmar1 enhancer is a gene therapeutic.

17. The method of claim 16, wherein the gene therapeutic is a recombinant Sigmar1 adenovirus infection in a tissue with a lowered ACE2 level.

18. The method of claim 9, wherein the gene therapeutic is a SIGMAR1 gene promotor.

19. The method of claim 9, wherein the gene therapeutic is one of an oligonucleotide therapy, a CAR-T therapy, a AAV transgene delivery, a gene editor, CRISPR-Cas9, and a universal donor cell therapy.

20. A method of treating coronavirus disease 2019 (COVID-19) patient comprising:
administering to the patient a pharmaceutically therapeutic dose of a therapeutic,
wherein the therapeutic includes a Sigmar1 enhancer, or any pharmaceutically acceptable salt, solvate, or prodrug thereof.
